(19) United States
(12) Patent Application Publication
(10) Pub. No.: US 2012/0179072 A1
(43) Pub. Date: Jul. 12, 2012

(54) SURGICAL DEVICE

(76) Inventor: Marc Kegreiss, Tubingen (DE)

(21) Appl. No.: 13/497,012

(22) PCT Filed: Sep. 22, 2010
(86) PCT No.: PCT/EP10/05806

§ 371 Date: Mar. 19, 2012

(30) Foreign Application Priority Data
Sep. 22, 2009 (DE) 10 2009 042 438.5

(51) Int. Cl. A61B 5/103; A61B 18/18

(52) U.S. Cl. 600/595; 606/32

(57) ABSTRACT

A surgical device for the treatment of biological tissue has a surgical instrument with at least one HF electrode and an HF surgical unit with an HF generator for generating an HF voltage and supplying an HF current to the HF electrode, and a control device for open-loop or closed-loop control of the HF generator. The surgical instrument has a movement detection device to detect a movement sequence of the surgical instrument and the HF surgical unit has an evaluation device for evaluating a movement sequence detected by the detection device. The movement detection device and the evaluation device are connected to each other, movement sequences of the surgical instrument detected by the detection device can be transmitted to the evaluation device, and the HF surgical unit is configurable by an evaluation of the movement sequences of the surgical instrument by the evaluation device.

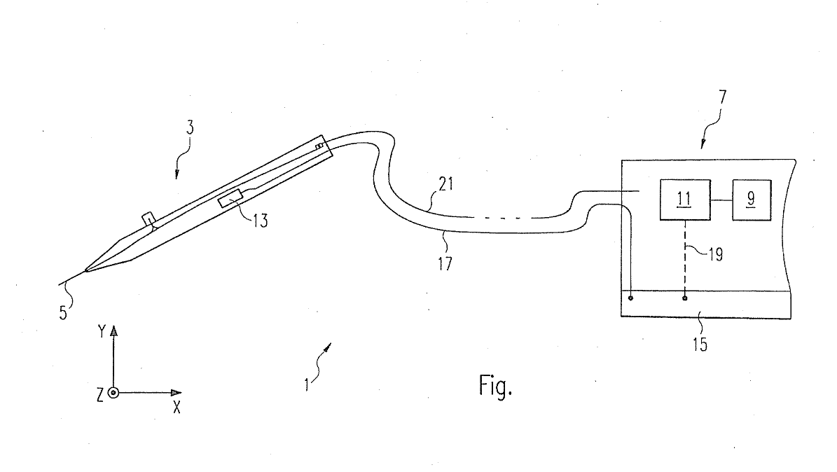

### FIELD OF THE INVENTION

Embodiments of the invention relate to a surgical device according and to the use thereof, a surgical instrument for use in a surgical device, an HF surgical unit for use in a surgical device and to a method for remote-controlled configuration of an HF surgical instrument.

### BACKGROUND

High-frequency (HF) surgery has been used for many years, both in human and in veterinary medicine, to coagulate and/or cut biological tissue. Suitable electrosurgical instruments are used to pass high-frequency current through the tissue to be treated so that it changes as a result of protein coagulation and dehydration. The tissue constricts such that the vessels are sealed and bleeding is stopped. A subsequent increase in current density causes an explosive evaporation of tissue fluid and a tearing of the cell membranes, with the tissue being fully separated.

Both bipolar and monopolar techniques are used for the thermal treatment of biological tissue. With monopolar arrangements, the HF current supplied by the HF generator to the electrosurgical instrument is applied to the tissue to be treated via a different electrode, with the current path through the body of a patient leading to an indifferent neutral electrode and from there back to the HF generator. The monopolar technique, by contrast, is of particular advantage for interstitial coagulation, in which a uniform (e.g., radially symmetrical) HF current is required across the tissue to be treated, i.e., extending over the target tissue. This enables, for example, tumors or metastases to be treated wherein the electrosurgical instrument suitable for monopolar coagulation is introduced into the tissue to be treated, for example into a tumor, and the destruction of the tumor is initiated (tumor devitalization) by the application of the high frequency current, i.e., by coagulation.

A surgical device of the type mentioned herein is known, for example, from German publication DE 10 2005 025 946. It preferably has a surgical instrument with at least one HF electrode and an HF surgical unit with one HF generator for generating an HF voltage and for supplying the HF current to the HF electrode. Furthermore, the HF surgical unit usually has a control device used for open-loop control or closed-loop control of the HF generator.

A disadvantage of the known surgical devices is that a change to user settings which have to be carried out on the HF surgical unit before or during an operation can only be made directly at the device itself. The surgical instrument is, however, in a sterile operating theatre, while the HF surgical unit is in a separate room and therefore cannot be adjusted by the operating doctor by himself.

An operating theatre nurse is therefore usually present in the adjoining room to make the settings on the HF surgical unit as instructed by the operating personnel. However, if the operating theatre nurse carries out the user settings on the HF surgical unit, this has the disadvantage that the operating personnel, in particular the doctor, must first communicate his configuration needs to the operating theatre nurse. The nurse must then ascertain the correct settings on the HF surgical unit and make them. This can lead to errors, especially as a result of communication problems.

Although known surgical devices, such as the VIO 300 D from the firm Erbe, have foot or finger switches, with which the HF surgical unit can be actuated from the operating theatre by the treating doctor, the functions of the foot or finger switches are limited to a maximum of two different configurations of the HF surgical unit. A further-reaching change to the user settings of the HF surgical unit from the operating theatre is not possible.

### SUMMARY

An object of the embodiments of the present invention is therefore to provide a surgical device and method for remotely configuring an HF surgical unit that allows a comprehensive change of settings of the HF surgical unit from the sterile operating theatre directly by the operating personnel.

To achieve this object, a surgical device that is used for the treatment of biological tissue and has a surgical instrument with at least one HF electrode, and a surgical unit with an HF generator are provided. The HF generator is used to generate an HF voltage and to supply the HF current to the HF electrode. The HF surgical unit further has a control device for open-loop or closed-loop control of the HF generator. The surgical device is characterized in that the surgical instrument has a movement detection device for detecting a movement sequence of the surgical instrument. A further characteristic of the surgical device is that the HF surgical unit comprises an evaluation device for evaluating a movement sequence detected by the movement detection device, with the movement detection device of the surgical instrument and the evaluation device of the HF surgical unit being connected to each other such that the movement sequences of the surgical instrument detected by the movement detection device of the surgical instrument can be transmitted to the evaluation device. The surgical device is further characterized by the fact that the HF surgical unit is configurable by evaluating the movement sequences of the surgical instrument by way of the evaluation device.

The surgical device proposed herein enables, in an advantageous manner, a large number of user settings to be made at the HF surgical unit directly from the operating theatre by the operating personnel. The movement detection device arranged in the operating theatre may be connected, for example, via a cable or by radio to the evaluation device to transmit detected movement sequences of the surgical instrument to the evaluation device. This removes the need for an operating theatre nurse to operate the HF surgical unit. To make any desired setting to the surgical unit, the operating doctor merely has to carry out a movement of the surgical instrument assigned to a setting which then triggers the corresponding configuration of the HF surgical unit. A movement of the surgical instrument may, for example, be a rotation or a lateral movement in one of the three spatial directions x, y or z. It may further be provided that a combination of movements is assigned to a particular setting of the surgical instrument. A rotational movement is followed, for example, by a lateral movement of the surgical instrument in order to bring about a desired assigned setting of the surgical instrument. A feature to distinguish between different movement sequences may also be the speed with which a movement of the surgical instrument is executed by the operating doctor.

In a particularly preferred surgical device, the movement detection device has a sensor unit, which preferably comprises at least one movement sensor, where the movement sensor may, for example, have at least one acceleration sensor. If the operating doctor executes a certain hand movement which is detected by the sensor unit, the correspondingly captured data are sent to the evaluation device and evaluated by it.

Particularly preferred is a surgical device characterized in that the evaluation device for evaluating at least one movement sequence of the surgical instrument has at least one pattern recognition algorithm. In this manner, the evaluation device can recognize the movement sequences transmitted by the movement detection device and assign a particular user setting to the recognized movement sequence. In this way, each movement sequence of the surgical instrument recognized by the evaluation device brings about an assigned user setting of the HF surgical unit.

Further preferred is a surgical device characterized in that a detected movement sequence brings about the execution of a stored treatment program, with the treatment program being stored in the HF surgical unit. Depending on the selected program, the HF generator then executes a corresponding closed-loop control of voltage or the like. This also makes it possible that the settings to be made are checked for their plausibility by the stored treatment program before they are implemented. To do so, the surgical device preferably has a mechanism for checking the setting of the HF surgical unit to be carried out. The mechanism can for example, be part of the evaluation device. Only when the HF surgical unit determines that the setting to be made is plausible is the configuration of the HF surgical unit changed. This prevents the operating doctor from performing an unconscious movement with the surgical instrument, and thus inadvertently making an unwanted change to the settings of the HF surgical unit.

A surgical device is further preferred which is characterized in that a visual or audible feedback device is provided. This is preferably in the region of the surgical instrument, i.e., in the operating theatre, and indicates that a setting of the HF surgical unit, initiated by a movement sequence, has been correctly recognized and implemented. For this purpose, the evaluation device preferably sends a signal back to the movement detection device which can be coupled to the feedback device.

To achieve the object of the embodiments of the present invention, a surgical instrument for use in a surgical device is further proposed. The surgical instrument has an HF electrode and a movement detection device for detecting a movement sequence of the surgical instrument. With regard to the advantages of the surgical instrument, attention is drawn to the above description for the surgical device.

The object is also achieved by an HF surgical unit for use in a surgical device where the HF surgical unit has an HF generator for generating an HF current and for supplying the HF current to the HF electrode of a surgical instrument, as well as an evaluation device for evaluating a movement sequence detected by a movement detection device of a surgical instrument. With regard to the advantages of the HF surgical unit, attention is drawn to the above description for the surgical device.

The object is also achieved by a method for remote-controlled configuration of an HF surgical unit for use in a surgical device. The method is characterized by the following steps: detection of a movement sequence of a surgical instrument by means of a movement detection device; transmission of the detected movement sequences to an evaluation device of the HF surgical unit; evaluation of the transmitted movement sequences of the surgical instrument by means of the evaluation device; and setting of a user configuration of the HF surgical unit assigned to the evaluated movement sequences.

Particularly advantageous is a method characterized in that the evaluation step comprises assignment of the movement sequence of the surgical instrument by way of a pattern recognition algorithm to a specific setting of the surgical device. In this way, the evaluation device can recognize the movement sequences transmitted by the movement detection device and assign a certain user setting to the detected movement sequence. As a result, each movement sequence of the surgical instrument detected by the evaluation device brings about a pre-determined user setting of the HF surgical unit.

A method is furthermore preferred in which the number of movement sequences that can be recognized by the evaluation device corresponds to the number of adjustable parameters of the surgical device. The method proposed herein advantageously enables a plurality of settings of the HF surgical unit to be made from the sterile operating theatre.

### BRIEF DESCRIPTION OF THE DRAWING

Hereinafter, exemplary embodiments of the invention are explained in greater detail with reference to drawing, which shows:

a schematic representation of a surgical device in accordance with the disclosed principles of the invention.

### DETAILED DESCRIPTION

The FIGURE shows a schematic representation of a surgical device 1 for the treatment of biological tissue. It has a surgical instrument 3 with an HF electrode 5, where the HF electrode 5 serves for treatment of the tissue of a patient, not shown, as explained above in detail.

The surgical device 1 further has an HF surgical unit 7, which comprises an HF generator 9 for generating an HF voltage and for supplying an HF current to the HF electrode 5. The HF surgical unit 7 moreover has a control device 11 for open-loop and closed-loop control of the HF generator 9.

The surgical instrument 3 has in accordance with the invention a movement detection device 13 to detect a movement sequence of the surgical instrument 3. Furthermore, the HF surgical unit 7 has an evaluation device 15 used to evaluate a movement sequence of the surgical instrument 3 detected by the movement detection device 13.

The movement detection device 13 of the surgical element 3 and the evaluation device 15 of the HF surgical unit 7 are connected to each other via a suitable line 17 such that movement sequences of the surgical instrument 3 detected by the movement detection device 13 can be transmitted to the evaluation device 15 of the HF surgical unit. It is, however, also conceivable for a wireless connection, in particular a radio link, to be provided between the movement detection device 13 and the evaluation device 15 so that data captured by the movement detection device 13, which represents a movement sequence of the surgical instrument, can be sent as radio signals to the evaluation device 15. To this end, the movement detection device 13 preferably has a corresponding transmitting station and the evaluation device 15 has a corresponding receiving station for the radio signals.

The movement detection device 13 preferably has a sensor unit that can have at least one movement sensor. The movement sensor can be realized by at least one acceleration sensor or the like to detect a movement of the surgical instrument 3. The technical design of the movement detection device 13 is not a crucial factor. The main point is that it recognizes a movement of the surgical instrument 3 and can transmit it to the evaluation device 15 in the form of data.

To achieve remote control of the HF surgical unit 7 by a certain movement of the surgical instrument 3, it is necessary for the treating doctor to first have knowledge of which movement sequences bring about which configuration changes to the HF surgical unit 7. To do so it is conceivable, for example, to provide an appropriate information sheet or the like in the operating theatre.

A movement of the surgical instrument 3 can, for example, be a rotational movement in a clockwise or counter-clockwise direction or also a lateral movement in one of the three spatial directions x, y, or z. It may further be provided that a combination of movements is assigned to a particular setting of the surgical instrument 3. A rotational movement must then be followed, for example, by a lateral movement of the surgical instrument 3 to implement a desired assigned setting of the surgical instrument 3. A feature to distinguish between different movement sequences can also be the speed with which a movement of the surgical instrument 3 is carried out by the operating doctor.

It should be expressly pointed out that the aforementioned movement of the surgical instrument 3 by the hand of the operator is not to be equated with the operation-specific movement of the surgical instrument, for example during a cutting procedure.

A predetermined movement/displacement of the surgical instrument 3 is preferably assigned to a certain treatment parameter which can be set by the HF surgical unit 7.

The HF surgical unit 7 can be individually programmable for optimum adaptation to appropriate interventions. The individual stored treatment programs can then be selected by an operator for the respective application. It is obvious that the stored treatment programs can also be standard programs specified by the manufacturer of the HF surgical unit 7.

It is conceivable, for example, to provide programs for different interventions such as plasma coagulation, cutting of tissue, devitalization of tissue and vaporization. The programs can moreover differ from one another in different cut depths, different types of tissue to be treated or different types of coagulation. The crucial factor is that a plurality of parameters is specified by different stored programs. An appropriate program can thus be selected directly from the operating theatre depending on the specialist field and the indication. The HF surgical unit then automatically sets the voltage values and/or other parameters to suit the selected program. The surgical device 1 proposed herein makes it possible for an operating doctor in the operating theatre to select the program required from a plurality of programs.

Furthermore, it is conceivable to make a selection between different predetermined execution modes of the HF surgical unit 7. These generate an automatically controlled appropriate cut quality to meet requirements, in particular due to suitable control of voltage. For specific types of tissue, such as especially adipose tissue, cutting can be carried out under water. For optimum cut quality, it is also possible to additionally use arc control. Furthermore, it is conceivable to employ a combination of voltage control and modulated waveforms. Special types of control can also be provided for bipolar cutting procedures.

Overall, it can be seen that the embodiments of the present invention do not bring about a remote-controlled direct setting of the HF generator 9, but rather a setting of the user's configuration of the HF surgical unit 7. The setting is made such that the HF generator 9 is only affected after the corresponding setting of the HF surgical unit 7 in accordance with the selected stored program. Accordingly, for example, voltage control, arc control or the like is then carried out.

Further, more modules can also be connected to the HF surgical unit 7, for example modules for argon plasma coagulation, for plume extraction, for rinsing pumps and the like. The modules are arranged, like the HF surgical unit 7, not in the sterile operating theatre but in an adjoining room, and thus cannot be actuated directly by the operating personnel. The expansion modules, not shown in the FIGURE, connected to the HF surgical unit 7 can then also be controlled by the detection of movement by the surgical instrument 3 of the type proposed here.

The proposed surgical device 1, in particular the HF surgical unit 7, can therefore be modular, where all modules can be controlled and configured by way of the movement detection device 13 and the evaluation device 15 interacting with it from the operating theatre. For this purpose, each of the modules can have its own evaluation device.

The HF surgical unit 7 preferably has automatic power metering for all control techniques, in particular for voltage control, arc control and power control. This advantageously dispenses with the need for the operating doctor to directly control voltage, current, frequency, etc., as these parameters are set automatically by the device. For this purpose, it is merely necessary to inform the device of which procedure is to be carried out and under which conditions. To do so, it is sufficient to select an appropriate stored individual program. This can be achieved by an appropriate movement of the surgical instrument 3 by the operator directly from the operating theatre.

In this way, the HF surgical unit 7 can be configured by an evaluation of the movement sequences of the surgical instrument 3 using the evaluation device 15, so that a plurality of user configurations of the HF surgical unit can be implemented directly from the sterile operating theatre.

To enable evaluation of the movement sequences transmitted by the movement detection device 13 of the surgical instrument 3, the evaluation device 15 preferably has at least one pattern recognition algorithm that recognizes movements and assigns the assigned user settings of the HF surgical unit 7. In this way, each movement sequence of the surgical instrument 3 recognized by the evaluation device 15 brings about a predetermined user setting of the HF surgical unit 3. It is preferably provided that each pattern recognition algorithm recognizes a movement, so that the number of recognizable movement sequences of the surgical instrument 3 is specified by the number of pattern recognition algorithms.

The nature of the movement sequences of the surgical instrument 3 is immaterial. It is conceivable, for example, for the operating doctor to “paint a letter in the air” or for the movement detection device 13 to recognize a real, surgical movement. Furthermore, it is also conceivable that a movement of the surgical instrument 3 in a longitudinal direction of the instrument is sufficient to bring about a certain configuration of the HF surgical unit 7.

As indicated in the FIGURE by the dashed line 19, the evaluation device 15 preferably interacts with the control device 11 such that a certain configuration is communicated to the control device 11 and is then implemented by the control device 11. It is also conceivable for the evaluation device 15 to be part of the control device 11. In this way, a plausibility check can be carried out before execution of the setting to be made, so that an inadvertent movement of the surgical instrument 3 by the operating doctor does not result in unwanted changes to the configuration of the HF surgical unit 7. For example, it is conceivable for an appropriate setting of the HF voltage to be applied to the HF electrode 5, or for the HF current to be set, to be communicated to the HF generator by the control device 11 so that the HF generator can then apply a corresponding voltage to the HF electrode via a connection 21 indicated in the FIGURE.

In a method for remotely configuring an HF surgical unit for use in a surgical device in accordance with the embodiments of the present invention, the following steps are performed:

First, the movement sequence of the surgical instrument 3 is detected by way of the movement detection device 13. Subsequently, the detected movement sequence is communicated to the evaluation device 15 of the HF surgical unit 7 in the form of data. The evaluation device 15 then performs an evaluation of the communicated movements of the surgical instrument 3 with the aid of pattern recognition algorithms. During the last step of the method, there is a change in the user setting of the HF surgical unit 7 as a function of the associated and evaluated movement sequences.

As stated above, the communicated movement sequences is preferably evaluated by the assignment of the movement sequences to a particular configuration of the HF surgical unit by means of a pattern recognition algorithm. The number of movement sequences recognizable by the evaluation device 15 thus preferably corresponds to the number of settable user configurations of the HF surgical unit 7. In this way, virtually any desired number of configurations of the HF surgical unit 7 can be set by way of the movement detection device 13 and the evaluation device 15.

It is crucial that the movement of the surgical instrument 3 first brings about a setting of the HF surgical unit and does not directly cause a change in output. Rather, it is only through the process of making a setting that a power control is triggered. It, therefore, does not bring about closed-loop control, but instead open-loop control of the HF surgical unit 7. In this way, it is possible that a detected/evaluated movement of the surgical instrument 3 is first confirmed before the user configuration of the HF surgical unit 7 is actually changed.

The method proposed herein and the corresponding surgical device 1 thus enable the user, i.e. in particular the operating doctor, to configure the HF surgical unit 7 directly from the sterile operating theatre in almost any way required, without the need for any additional personnel in the effective range of the HF surgical unit 7. Virtually any desired information can be exchanged between the HF surgical unit 7 and the surgical instrument 3 via a suitable communication path shown in the FIGURE by line 17. This enables among other things, visual or audible feedback to the operating doctor on whether a command to change a setting communicated by a movement is firstly recognized by the evaluation device 15 and secondly has been carried out.

As already indicated above, the movement carried out by the operating doctor to bring about a change in setting can be of almost any desired form. It is conceivable, for example, to have a movement along a longitudinal axis of the surgical instrument 3. Furthermore, it is also conceivable to provide a movement sequence in a direction orthogonal to the longitudinal axis. It is further conceivable to have a rotating movement of the surgical instrument 3, to which a particular configuration of the HF surgical unit 7 is assigned. The movement sequences are then detected with the transmitted values of the sensor by pattern recognition algorithms in the evaluation device 15, as already explained above.

It has been found that almost all settings of the surgical device 7 can be made directly from the sterile area. The number of setting mechanisms is limited only by the gradation of the pattern recognition algorithms. A plurality of possible commands that can be transmitted to the HF surgical unit 7 is usually provided.

1-13. (canceled)

14. A surgical device for the treatment of biological tissue, said device comprising:
a surgical instrument with at least one high frequency (HF) electrode; and
an HF surgical unit with an HF generator for generating an HF voltage and for supplying HF current to the HF electrode, said HF surgical unit comprising a control device for open-loop or closed-loop control of the HF generator, wherein:
the surgical instrument has a movement detection device for detecting a movement sequence of the surgical instrument,
the HF surgical unit has an evaluation device for evaluating the movement sequence detected by the movement detection device,
the movement detection device and the evaluation device are connected to each other and the movement sequence of the surgical instrument detected by the movement detection device is transmitted to the evaluation device, and
the HF surgical unit is configurable by an evaluation of the movement sequence of the surgical instrument by via the evaluation device.

15. The surgical device according to claim 14, wherein the movement detection device has a sensor unit with at least one movement sensor.

16. The surgical device according to claim 14, wherein the movement detection device has a sensor unit with at least one acceleration sensor.

17. The surgical device according to claim 14, wherein the evaluation device has at least one pattern recognition algorithm for evaluating at least one movement sequence.

18. The surgical device according to claim 14, wherein each movement of the surgical instrument recognized by the evaluation device results in a predetermined user setting for the HF surgical unit.

19. The surgical device according to claim 14, wherein a detected movement sequence brings about execution of a stored treatment program.

20. The surgical device according to claim 14, further comprising means for verifying the setting of the HF surgical unit to be made.

21. The surgical device according to claim 14, further comprising a visual or audible feedback device.

22. A surgical instrument for use in a surgical device for the treatment of biological tissue, said instrument comprising:
an HF electrode; and
a movement detection device for detecting a movement sequence of the surgical instrument, wherein:
the surgical device comprises an HF surgical unit with an HF generator for generating an HF voltage and for supplying HF current to the HF electrode, the HF surgical unit comprises a control device for open-loop or closed-loop control of the HF generator,
the HF surgical unit has an evaluation device for evaluating the movement sequence detected by the movement detection device,
the movement detection device and the evaluation device are connected to each other and the movement sequence of the surgical instrument detected by the movement detection device is transmitted to the evaluation device, and
the HF surgical unit is configurable by an evaluation of the movement sequence of the surgical instrument by via the evaluation device.

23. An HF surgical unit for use in a surgical device for the treatment of biological tissue, said HF surgical unit comprising:
an HF generator for generating an HF current and for supplying the HF current to an HF electrode of a surgical instrument and comprising an evaluation device for evaluating a movement sequence of the surgical instrument detected by a movement detection device within the instrument, wherein:
said HF surgical unit comprises a control device for open-loop or closed-loop control of the HF generator,
the movement detection device and the evaluation device are connected to each other and the movement sequence of the surgical instrument detected by the movement detection device is transmitted to the evaluation device, and
the HF surgical unit is configurable by an evaluation of the movement sequence of the surgical instrument by via the evaluation device.

24. A method of remotely configuring an HF surgical unit for use in a surgical device for the treatment of biological tissue, said method comprising:
inputting a detected movement sequence of a surgical instrument into an evaluation device of the HF surgical unit;
evaluating the detected movement sequence of the surgical instrument at the evaluation device; and
setting a user configuration of the HF surgical device based on the evaluated movement.

25. The method according to claim 24, wherein the evaluation step evaluates the movement sequence using pattern recognition.

26. The method according to claim 24, wherein a number of movement sequences recognizable by the evaluation device corresponds to a number of settable user configurations.

27. The method according to claim 24, wherein the detected movement sequence brings about execution of a stored treatment program.

28. The method according to claim 24, further comprising verifying the setting of the HF surgical unit to be made.

29. The method according to claim 24, further comprising providing visual feedback of the setting change.

30. The method according to claim 24, further comprising providing audible feedback of the setting change.
